(19) United States
(12) Patent Application Publication
(10) Pub. No.: US 2026/0325171 A1
(43) Pub. Date: Oct. 22, 2026

(54) System and Methods for Minimally Invasive Removal of Implanted Devices

(71) Applicant: NEURONOFF, INC., Cleveland, OH (US)

(72) Inventors: Amelia HOWE, Cleveland, OH (US); John WARDLE, Cleveland, OH (US); Stephan NIEUWOUDT, Cleveland, OH (US); Manfred FRANKE, Cleveland, OH (US); Morgan MCGAUGHEY, Cleveland, OH (US); SHAHER AHMAD, Cleveland, OH (US); Jacob CONARD, Cleveland, OH (US)

(73) Assignee: NEURONOFF, INC., Cleveland, OH (US)

(21) Appl. No.: 19/541,536

(22) Filed: Feb. 17, 2026

Related U.S. Application Data
(63) Continuation-in-part of application No. 19/468,229, filed on Feb. 3, 2026.
(63) Continuation-in-part of application No. 19/456,700, filed on Jan. 22, 2026.
(63) Continuation of application No. 18/561,425, filed on Nov. 16, 2023.
(63) Continuation-in-part of application No. 18/285,349, filed on Oct. 2, 2023, now Pat. No. 12,642,575.
Application No. 18/285,349 is a continuation-in-part of application No. 18/278,160, filed on Aug. 21, 2023.
Application No. 18/278,160 is a continuation-in-part of application No. 18/026,705, filed on Mar. 16, 2023.
(60) Provisional application No. 63/167,836, filed on Mar. 30, 2021.
(60) Provisional application No. 63/171,780, filed on Apr. 7, 2021.
(60) Provisional application No. 63/184,656, filed on May 5, 2021.
(60) Provisional application No. 63/226,458, filed on Jul. 28, 2021.
(60) Provisional application No. 63/236,678, filed on Aug. 24, 2021.
(60) Provisional application No. 63/306,896, filed on Feb. 4, 2022.

(51) Int. Cl. A61B 17/34; A61B 17/00

(52) U.S. Cl. CPC A61B 17/3468; A61B 2017/00349; A61B 2017/3454; A61B 2017/3456; A61B 2217/005; A61B 2217/007

(57) ABSTRACT

A system for minimally invasive removal of small implanted devices, without surgery or open cut down, by blunt separation from tissue. An introducer tool penetrates tissue and a removal needle with rounded elements attaches the implant without cutting. Force limiting and smoothing also prevents implant from shearing during removal.

### CROSS-REFERENCE TO RELATED APPLICATIONS

This application is a continuation in part of U.S. patent application Ser. No. 19/468,229 filed Feb. 3, 2026, This application is a continuation in part of U.S. patent application Ser. No. 19/456,700 filed Jan. 22, 2026. This application is a continuation in part of U.S. patent application Ser. No. 19/191,595 filed Apr. 28, 2025. This application is a continuation of U.S. patent application Ser. No. 18/561,425 filed Nov. 16, 2023. This application is a continuation in part of U.S. patent application Ser. No. 18/285,349 filed Oct. 2, 2023.

U.S. patent application Ser. No. 18/285,349 is a continuation in part of US patent application Ser. No. 18/278,160 filed Aug. 21, 2023. U.S. patent application Ser. No. 18/285,349 is a continuation in part of U.S. patent application Ser. No. 18/026,705 filed Mar. 16, 2023.

U.S. patent application Ser. No. 18/561,425 is a National Phase entry of international application PCT/US2022/029918 filed May 18, 2022.

U.S. patent application Ser. No. 18/285,349 is a National Phase entry of international application PCT/US2022/029918 filed May 18, 2022.

U.S. patent application Ser. No. 18/285,349 is a National Phase entry of international application PCT/US2022/020652 filed Mar. 16, 2022. PCT/US2022/020652 claims priority to, and the full benefit of, U.S. provisional application No. 63/306,896 filed Feb. 4, 2022.

PCT/US2022/029918 claims priority to, and the full benefit of, U.S. provisional application No. 63/236,678 filed Aug. 24, 2021. PCT/US2022/029918 claims priority to, and the full benefit of, U.S. provisional application No. 63/226,456 filed Jul. 28, 2021. U.S. patent application Ser. No. 18/026,705 is a National Phase entry of international application PCT/US2021/033265 filed May 19, 2021. U.S. patent application Ser. No. 18/278,160 is a National Phase entry of international application PCT/US2021/033007 filed May 18, 2021. PCT/US2022/020652 claims priority to U.S. provisional application No. 63/184,656 filed May 5, 2021. PCT/US2022/020652 claims priority to U.S. provisional application No. 63/171,780 filed Apr. 7, 2021. PCT/US2022/020652 claims priority to U.S. provisional application No. 63/167,836 filed Mar. 30, 2021 all of which are incorporated by reference herein in their entirety.

### BACKGROUND OF THE INVENTION

### 1) Field of the Invention

The field of the invention relates to small devices implanted in bodies to serve as leads or electrodes, such devices including helical wire rope structures.

### SUMMARY OF THE INVENTION

The invention here comprises a system for minimally invasive removal of a small implant from bodily tissue may comprise a removal needle comprising a shaft, a tip, and an attachment tool located at or near the tip. The attachment tool may be configured for attaching to and removing an implant intact, without requiring a surgical procedure. The removal needle may further comprise an interior channel, and the attachment tool can be configured to pull the implant through the interior channel. The attachment tool may be selected from the group of tools consisting of a hooking slot, a hook, a corkscrew, a point, a cutout, and multiple prongs. The removal needle may comprise a side port connected to the interior channel near the distal end, and the attachment tool may be exposed to the implant through the side port. The removal needle tip may be sharp and configured to penetrate the tissue in which the implant is implanted. Alternatively, the removal needle tip may be blunt or rounded.

The system may further comprise an introducer tool that comprises a penetrating tip and an introducer channel that is sized to allow insertion of the removal needle to the implant. The system may further comprise an introducer tool engagement rod that connects the introducer tool to a handle of the removal needle.

The system may further comprise a connection port to an external system. The external system may be a suction device, or may be a fluid source.

The system may further comprise a force limiting fuse and/or a force smoothing device that is connected to or embedded within the removal needle.s.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIGS. 1A-1G show two embodiments of removal needles, each comprising an attachment tool which is a hooked slot and having a sleeve. Embodiments may have a catch (as shown in FIGS. 1G-1F) or be formed without a catch (as shown in FIGS. 1A-1D).

FIGS. 2A and 2B are images obtained by fluoroscopy of a removal needle with an attachment tool which is a corkscrew, shown engaged with a helical wire rope structure in an animal.

FIGS. 3A and 3B show a removal needle where the attachment tool is a hooking slot and where the removal needle has an interior channel and a solid tip or a plug.

FIGS. 4A-4C depict an embodiment of the system in which the removal needle, with a handle, has an attachment tool comprising a rounded point, and engages with a helical wire rope structure.

FIGS. 5A-5C depict an embodiment of a portion of the removal needle with an attachment comprising rounded points and cutouts for capturing a helical wire rope structure.

FIGS. 6A-6C depict an embodiment of the removal needle and handle, and with a blunt tip, a side port, and an attachment tool comprising a corkscrew. FIG. 6D shows a portion of a removal needle where a corkscrew exits the tip of the needle and engages the helical wire rope structure.

FIG. 7A shows an embodiment of the system with a handle for a removal needle wherein the attachment tool comprises three hooked prongs. FIG. 7B is a transverse section view of the removal needle at line A-A and 7C is a transverse section view of the three prongs at line B-B.

FIG. 8A shows an embodiment of the removal needle with a handle and removal needle and attachment tool comprising two prongs which have expanded after being exposed. FIG. 8B is a longitudinal section view of the removal needle at line C-C with the attachment tool contained in the sheath and 8C is a similar view with the attachment tool exposed by withdrawal of the sheath.

FIG. 9A shows an embodiment of a portion of the removal needle in which the attachment tool comprises three inwardly turning hooked prongs with rounded ends having two segments, and has a dotted line showing a ninety degree axis to the long arm of one of the hooked prongs. FIG. 9B shows inwardly facing prongs with rounded ends in three segments in the shape of hooks. FIG. 9C is a similar view of an embodiment with two outwardly facing hooked prongs.

FIG. 10A shows one embodiment of the system which includes an introducer tool with a penetrating tip through which is inserted a removal needle with the attachment tool comprising three prongs. FIG. 10B is a transverse section of this same embodiment with the needle and long arms of the prongs pulled back into the introducer tool. FIG. 10C shows an introducer tool engagement rod linking the introducer tool to the handle of the removal needle.

FIG. 11 is a longitudinal section view of an embodiment of a force limiting fuse and force smoothing device.

FIG. 12 is a chart showing removal forces and times with and without force smoothing.

FIGS. 13A-13D are a sequence of images of a removal needle with a hooking slot engaging a bunching anchor in a ballistics gel and beginning the removal of the helical wire rope structure by unzipping.

FIG. 14 shows a wire hook tool.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

The present disclosure describes a removal system and methods for “minimally invasive removal,” which means removal of small implanted devices without a surgical procedure or other open cut down at the skin. The system limits the maximum removal forces acting upon a helical wire rope structure or other implant placed near delicate anatomical structures such as nerves, arteries and other vessels inside a living body. The system and method enable the blunt separation of an implant from surrounding encapsulating tissue by mechanically connecting to the implant and carefully pulling the implant from the tissue, all being removed either through or jointly with a removal needle. In one or more embodiments the system incorporates a force smoothing component as well as a force limiting component to ensure neither the implant nor nearby delicate anatomical structures are damaged during the blunt removal process. Blunt removal carefully separates a tissue layer from other tissues or tissue planes following microscopic and macroscopic anatomical lines, but does not cut. The system and method enable a helical wire rope structure to unzip from surrounding tissues with a layer of encapsulation without cutting, ripping or breaking the helical wire rope structure. The system and method enable a helical wire rope structure to release from surrounding tissues with a layer of encapsulation without cutting, ripping or breaking the helical wire structure that may not feature unzipping.

The system and method enable any other implant to release from surrounding tissues with a layer of encapsulation without cutting, ripping or breaking the non-helical wire rope structure that may not feature unzipping. Despite the ability of the clinician to use the device without surgical incision, the system and method can be used with an open incision if the clinician so desires.

The system may employ a specialized removal needle 33 with an attachment tool (whose embodiments may be selected from the group consisting of a hooking slot 52, corkscrew 54, prongs 62, hooks 63, 67-68, and rounded points 82 and cutouts 59) which is inserted through an introducer tool 64, 64A and/or or is used by itself with an exterior sleeve 53.

As used herein, “attachment tool” can mean any one or a combination of the above embodiments. The attachment tool in several embodiments is at the distal end of a rod 54A, and the rod pushes the attachment tool through the removal needle and to the implant. The attachment tool may employ rounded surfaces, even when the term “point” is used, so as not to cut the implant when the clinician is seeking to secure it with the attachment tool.

The system herein is configured for minimally invasive removal of a small implanted device 1 from within the body by (1) advancing the introducer tool 64, 64A to the location of the implant, (2) inserting the removal needle 33 through the introducer tool and mechanically engaging the attachment tool with the implant at any portion along its length by means of the removal needle's attachment tool and without cutting portions of the implant off from the implant during the mechanical attachment process, (3) pulling out the small implant with the removal needle either (a) through the introducer tool while the introducer tool stays in place as a channel and the implant passes though the inside of the introducer tool, or (b) together with the introducer tool and thereby pulling a portion of the implant into the removal needle while the majority of the implant is pulled out once the introducer tool and removal needle are outside the body.

In one embodiment the system comprises the following elements:
a. an introducer tool (e.g., within a range of 20 Ga (0.9 mm OD) to 7 Ga (4.5 mm OD)),
b. a removal needle (e.g., within a range of 22 (0.7 mm OD) to 8 Ga (4.2 mm OD)), with an attachment tool at or near the removal needle's tip,
c. a handle along the removal needle and/or introducer tool with one or more buttons to either (a) move the introducer tool back and forth along the tip and shaft of the removal needle, thus exposing the removal needle from the introducer needle and thereby expose it to the tissue of the body after the introducer tool has pierced the skin, or (b) move the attachment tool in and out of the removal needle, thereby exposing the attachment tool to the tissue and the implant inside the body

In several embodiments, the introducer tool punctures the skin and provides the channel for the removal needle to pass through to reach the implant inside the body. The length of the introducer tool may be marked with depth measurements (e.g., mm and cm scale) to help the clinician see where the introducer tool is located within the body. The introducer tool, and marks on it, may be radio opaque. The introducer tool features a handle 64 independent from or in a mechanical combination with the removal needle's handle 57. When the introducer tool handle 64 is independent handle of the removal needle's handle then the introducer tool's handle may hold and guide the introducer tool into the body. There is a region designed to secure the handle with tape or a clip to the body in order to mechanically anchor the introducer tool with the body and be able to independently move the removal needle's shaft in and out of the introducer tool without moving the introducer tool at the same time. The handle 64 may be screwed to a Luer Lock that is part of a rod 78. This way the introducer tool handle 64 can be mechanically locked via button 77 to move either together with the introducer needle 33 or to move along the needle 33, thereby letting the needle 33 pass freely into the body after having pierced a puncture wound and having been unlocked via the button 77 thereafter.

The removal needle may be sharp or blunt. A removal needle with a blunt tip can be inserted through a sharp introducer tool to puncture the skin and any tissues on the path to the implant.

A blunt introducer tool may be used additionally to advance the blunt or sharp removal needle near delicate anatomical structures. Alternatively, liquid based dissection via a smaller diameter needle (smaller than the introducer tool) may be used to create a channel through which a blunt introducer tool may be placed over the smaller diameter liquid injection needle. When using a blunt introducer tool, an additional smaller diameter needle (smaller than the introducer tool but not pictured) can be used to inject a liquid (e.g., saline, lidocaine) continuously or intermittently while the removal needle is advanced into the body. Once the introducer tool is nearby or in close proximity or adjacent or in mechanical contact with the implant, then either one of the sharp needle, sharp or blunt introducer tool may be exchanged for the removal needle.

In one embodiment, the removal needle has a Luer Lock at or in the handle 57, or the introducer tool has a Luer Lock at the engagement rod 78 to interface with the handle 64 of the introducer tool. After advancing the introducer tool through the skin and tissue towards the implant, the engagement rod may disengage from the handle enabling the free relative movement of the removal needle 33 relative to the introducer tool 64, 64A. The introducer tool may be left in place near the implant, thus providing a channel for the removal needle to be introduced through the introducer tool into the body, or exiting the introducer tool near the implant.

In another embodiment the introducer tool (e.g., 3 to 5 cm length) is used only to puncture the skin and the removal needle comprises a mechanically stiff backing which itself has a sticky surface. The short introducer tool is radio opaque and allows the clinician to visualize where the skin is which in tum helps seeing how the removal needle will move on fluoroscopy as the sticky surface of the mechanically stiff backing secures the needle to the skin.

In one embodiment with the introducer tool, a lock mechanism on the handle locks the removal needle and its attachment tool in place within the introducer tool so that the removal attachment is not exposed until the physician desires. In this way the introducer tool punctures the skin, then the button is pushed to unlock the removal attachment which then allows for free movement in and out of the introducer tool.

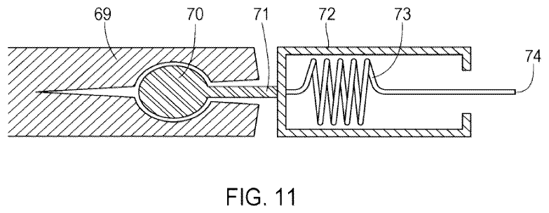

In one embodiment, the system comprises a removal needle handle 57 to control the attachment tool, an introducer tool configured to allow the removal needle/attachment tool to be passed through the introducer tool and into the body. The attachment tool for the implant operates so that mechanical forces of at least 2 N and up to 50 N may be transferred from the removal needle to the implant. The force limiting fuse and/or force smoothing device shown in FIG. 11 helps to limit the maximum forces to stay below a maximum such as 50 N defined by the elements 69 and 70.

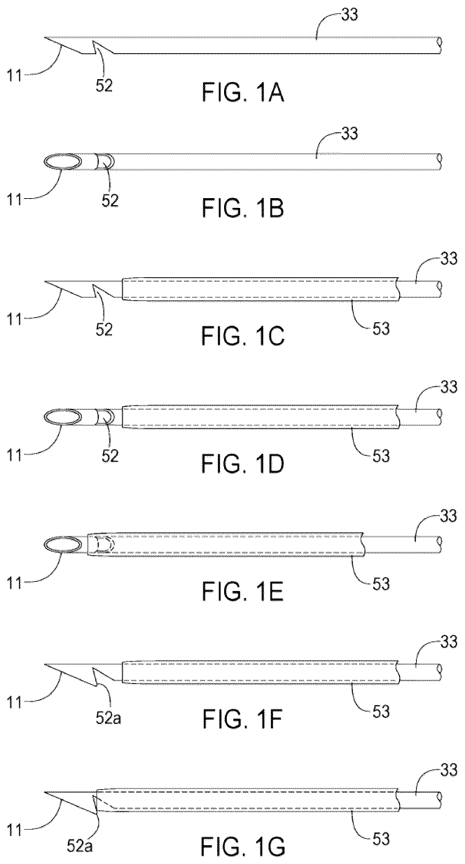

An embodiment of the removal needle 33 with the attachment tool comprising a hooking slot 52 with round edges (rounding to avoid cutting action on the implant) is shown in FIGS. 1A, 1B, 1C and 1D, and this embodiment can be used with or without the introducer tool. A sleeve 53, shown in FIGS. 1C and 1D, may be used to secure the implant attached by the hooking slot, as shown by the movement of the sleeve over the hooking slot in FIG. 1E. FIGS. 1F and 1G depict an embodiment with an optional catch 52A to prevent shear forces being applied to an implant when the sleeve 53 is being pushed over the hooking slot 52. The width of the needle at the catch 52A is greater than the width of the removal needle shaft.

Other similar embodiments may be used as well to prevent shear caused by the sleeve totally enclosing the hooking slot. An opening of approximately 0.25 mm may prevent shear forces applied to the implant during the attaching and extraction procedure. Various sleeve lengths can be used to cover the hooking slot during insertion or during repositioning inside the body before hooking onto the implant. The sleeve can be used to partially or fully cover the hooking slot in the needle by moving the sleeve across the implant once it has been attached by the hook. This method provides the clinician with a reliable engagement while pulling the implant out of the body once hooked. The sleeve is pushed forward to cover the hooking slot (and side port 61) or pulled back to expose the hooking slot (and side port) as needed. In one embodiment, the sleeve itself has a flexible section along its length to enable a force smoothing and maximum force limiting effect when the clinician mechanically engages the locking mechanism, thereby reducing the risk of cutting or shearing the implant while mechanically engaging and holding it. These embodiments may allow a linear transfer of force into and out of the body using the clinician's tactile feedback. There may be embodiments in various lengths and diameters.

The force limiting fuse shown in FIG. 11 formed by the socket 69 and ball 70 limits the holding force applied to an implant to avoid cutting or shearing the implant before or during the removal process and may limit the holding/crushing force applied between the needle 33 and the sleeve 53.

The goal of the socket 69 and ball 70 is to enable securing the implant within the removal tool to enable the transfer of a torque and/or a force to disengage the implant from the surrounding tissue, and then then to remove the implant from a body by pulling the implant without creating an unreasonably high risk of cutting or shearing off elements of the implant during the engagement and/or pulling process.

The removal needle herein may have blunt or rounded edges, to enable blunt separation of the implant from the tissue (not cutting tissue from the body as with some current devices). The force limiting and smooth features discussed elsewhere herein also prevent breaking, cutting, or shearing of the implant and may also prevent removing too much tissue.

The sleeve 53 may be pushed forward and/or pulled backwards with a mechanism similar to that of a ball point pen cartridge locked in an outside and an inside position using a button operated locking mechanism. This enables the clinician to pull the sleeve back when the removal needle is near an implant and to use the sleeve to securely hook the implant for one-handed operation for the clinician. Once engaged, this embodiment keeps the needle in an advanced position or allows it to be retracted. Here the spring pressure may hold the hook on the implant, thereby exerting the sufficient amount of hooking pressure to retrieve a chronically implanted device with tissue encapsulation. This also limits the risk of cutting or shearing the implant during the process of mechanically locking the implant or the process of mechanically dislodging the implant from the encapsulation tissue and the process of mechanically removing the implant from the body. One example for such a mechanism is found in U.S. Pat. No. 2,734,484 to O'Sullivan, and other mechanisms are currently available.

The removal needle with a hooking slot may have markers for optimized fluoroscopic and/or ultrasound visualization or to allow depth assessments during the procedure. The sleeve on the outside of the needle may have markers visible without additional visualization, thereby providing depth assessments during the removal procedure. The clinician may use the markers, e.g., all along the needle and spaced at regular centimeter intervals on the removal needle, or being placed only at distinct locations such as 1 cm from the beveled tip, in the middle (e.g. 3 cm from the first marker) and 1 cm from the end near the plastic connector (e.g. Luer Lock or Luer Slip).

The removal needle with a hooking slot may be hollow or may be solid. The hollow needle embodiment with an interior channel may be attachable to an external system for the introduction of fluids or gases, or it may be connected to suction for the removal of liquids, gases or to aid with the hooking to the implant. In embodiments where the removal needle is not hollow but solid, the removal needle is essentially a rod. The removal needle with a hooking slot, or, respectively, a hooked rod, may be attached to a handle 57 using a Luer Lock or different interface to provide more mechanical stability to the clinician. The handle 57 of the removal device may have an opening to accommodate an engagement rod 78, reversibly secured with a spring operated button, screw or clip 77, such that a set distance between an introducer needle and the handle can be achieved. This can be accomplished by securing an introducer tool 64 onto the attachment rod with optional Luer lock (or Luer Slip), thereby being able to move the introducer tool with the handle of the introducer tool and removal needle all at once. To utilize the introducer tool, the user locks the removal needle at a set distance such that the tip of the removal needle is fully hidden inside the introducer tool before creating a puncture wound in the skin nearby the implant location. Once the skin is pierced with the introducer tool 64A, the button 77 is pushed (or screw rotated) to mechanically unlock the attachment rod from the handle, thereby allowing the rod to slide deeper into the handle of the removal needle handle. This enables the removal needle 33 to penetrate from the front tip of the introducer needle 64A without moving the introducer needle further into the body and enables a blunt approach towards the implant with the removal needle 33 prior to pushing the attachment tool 62 out of the removal needle to engage with the implant. Once the implant is engaged, the removal needle 33 may be pulled through the introducer needle 64A or may be pulled out at once together with the introducer tool handle 64, thereby removing the implant from the body. The wound may be closed by stitch, glue or steri-strip.

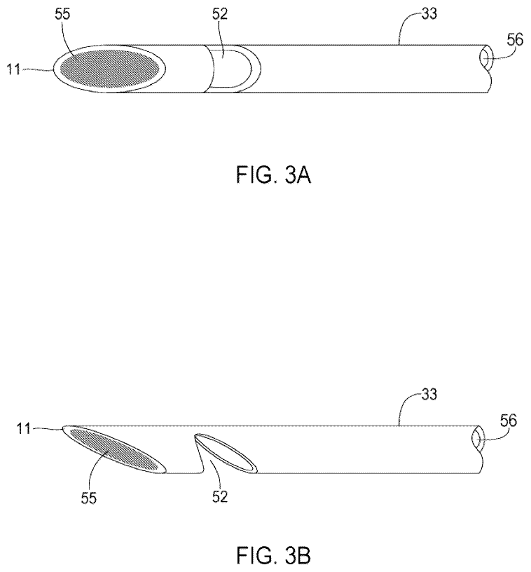

The removal needle with a hooking slot may have a thinning slit and rounded edges on the slit as shown in FIG. 3B near the beveled end of the needle which reduces the slit diameter while offering rounded edges. This prevents cutting when attaching the implant during the pulling process. In one implementation, the diameter of the slit may start at 1.5 mm width and narrow down to a diameter of 0.3 mm to facilitate attaching an implant (helical wire rope structure with un-deformed diameter about 0.8 mm) based on a wire rope of approximately 0.25 mm diameter. The slit may further be covered on all edges with a soft cover material such as a foam or flexible rubber to further aid with a reduction in risk of cutting the implant when pulling on it. One embodiment of a soft covering is made by applying a rubbery paint on the slit or dipping the slit into a rubbery paint, optionally followed by airbrushing off any excess rubbery paint from the shaft of the needle as well as the hooking slit itself. The edges of the slit are generally rounded so as to not create force/pressure points on the implant during the pulling or twisting process. The system disclosed herein separates the implant from encapsulation but does not cut the implant from encapsulation in order to avoid cutting the implant and leaving a portion of it behind.

In general, the removal needle with a hooking slot 52 (in its various embodiments) may be used to poke (stab) next to an implant, through an implant, and/or into a bunched-up anchor of an implant, and it may either be pulled on, suctioned into, or slightly rotated to improve with the hooking of the implant before pulling out the implant with the removal needle with a hooking slot.

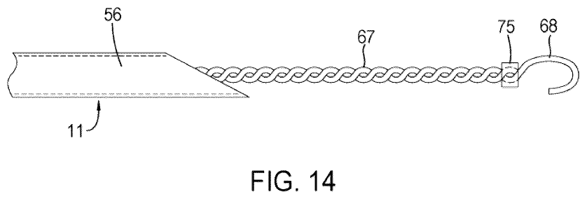

Furthermore, a wire hook tool 67 (in one embodiment a one-stranded wire folded back on itself) as shown in FIG. 14, is configured to passed through the introducer tool and the open tip of the removal needle. Optionally, the wire hook tool is pushed through the removal needle as shown in FIGS. 1A-1E with a hooking slot to increase the probability of hooking onto and securing the implant mechanically before pulling it out for removal.

In one embodiment the wire hook tool 67 comprises at least one hook 68 on the end of long wires small enough to remain minimally invasive but large enough to not plastically deform during removal. In one embodiment, the wire hook tool can be made with stainless steel due to its high yield strength and mold ability. During removal, a large amount of stress is put along the body of the wire hook tool. To prevent plastic deformation in this area, the body of the wire hook tool can further comprise wrapping with additional wires which increase the yield strength of the body while not substantially increasing the size of the tool. When removing the helical wire rope structure, the shape of the wire hook tool increases the failure force needed to sever the helical wire rope structure (discussed herein) and therefore has a higher success rate when removing a helical wire rope structure. The volume that can be removed by the device is the volume inside the wire hook tool multiplied by the length of the body of the device that is directly above the hook.

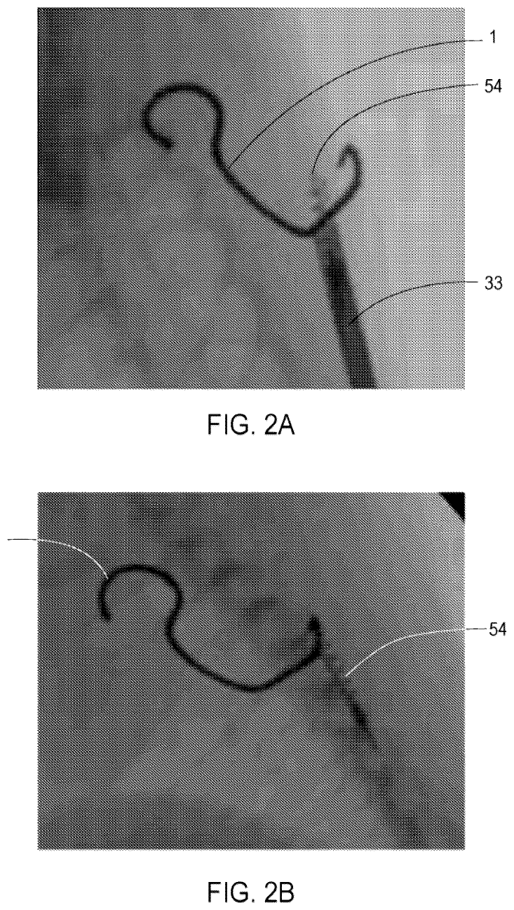

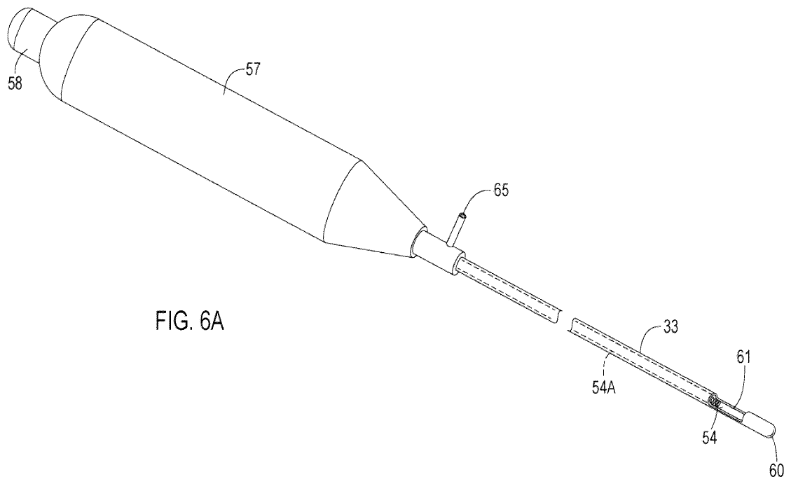

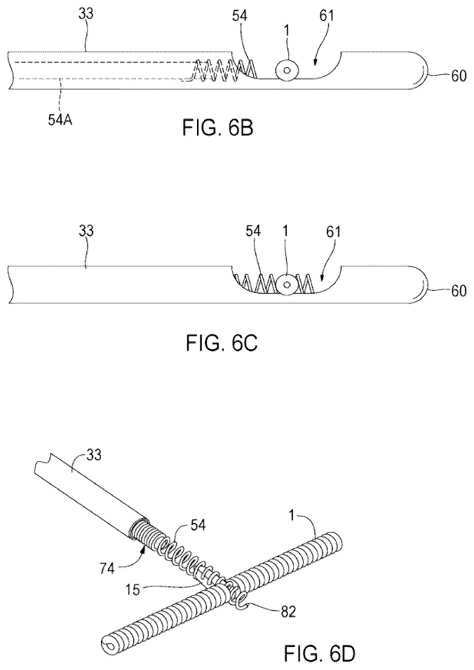

The attachment tool, in one embodiment, comprises a corkscrew 54 on a rod 54A or a corkscrew on a wire, as shown in FIGS. 2A-2B and FIGS. 6A-6D. The corkscrew has the ability to be pushed through the interior channel of the removal needle (e.g. removal needle inner diameter 1.5 mm, corkscrew and corkscrew driver outer diameter 1.45 mm) to the location of a helical wire rope structure to mechanically connect by screwing the corkscrew into the helical wire rope structure, thereby locking the two together and then allow for a sufficient pulling force (e.g. 1 to 2 Newton) a pulling wire connected to the corkscrew which in turn pulls on the helical wire rope structure with said pulling force (e.g. 1 to 2 N), allowing the helical wire rope structure to be released from the tissue and pulled out via the cannula together with the corkscrew (FIGS. 2A & 2B).

FIG. 2 shows fluoroscopic images of the attachment tool as a corkscrew 54 and a corkscrew driver 54A configured to transfer torque applied from the outside of the body to the corkscrew inside the body to mechanically interconnect with the chronically implanted helical wire rope structure. FIG. 2A shows the combination of the corkscrew with rod (the rod obstructing the view on the lead that is attached to the corkscrew) which is partially visible on fluoroscopy in FIG. 2B. This embodiment of the corkscrew and rod has an outside diameter of 1.45 mm in order to fit through the removal needle of 1.5 mm inner diameter, but other sizes including smaller versions are also possible.

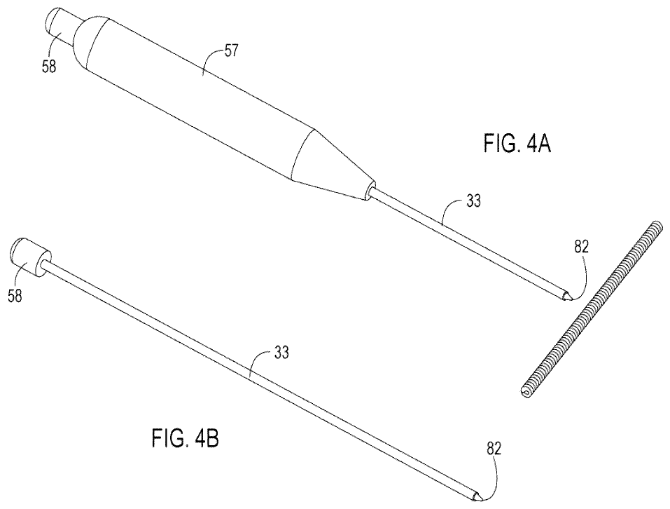

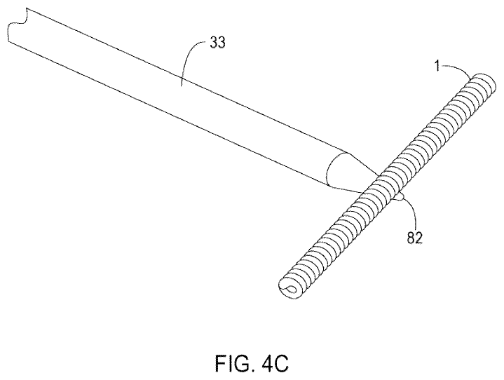

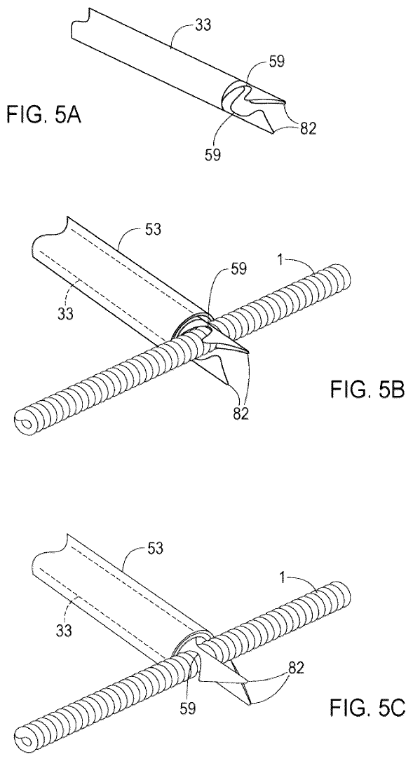

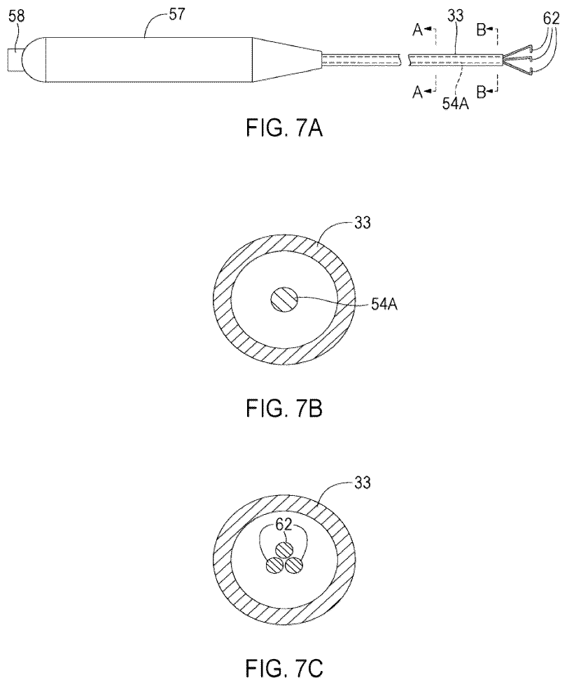

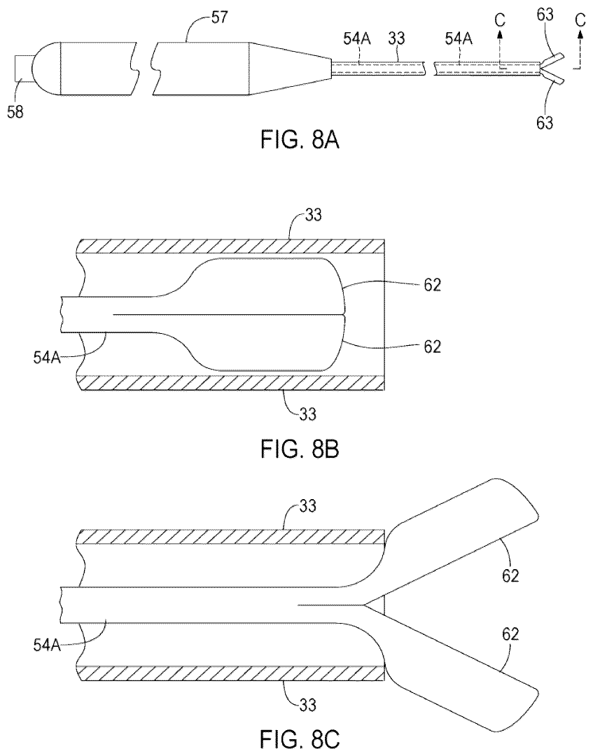

Embodiments of the attachment tool as prongs and hooks are depicted in FIGS. 7A-7C, 8A-8C, 9A-9C and 10A-10C. Another embodiment of the attachment tool comprises two prongs as shown in FIGS. 8A-8C. Yet another embodiment of the attachment tool as a rounded point 82 is depicted in FIGS. 4A-4C, and rounded points 82 with also rounded cutouts 59 are shown in FIGS. 5A-5C. All of these embodiments may be inserted through the introducer tool which has already been placed adjacent to the implant. The corkscrew may be operated by button push mechanically where a linear button push translates into a linear motion of the corkscrew at the same time as a rotational motion is turning the corkscrew. The corkscrew may be operated by an electrical driver, e.g., inside the handle 57 to aid with the turning of the corkscrew. A button on the handle may engage the electrical driver to help lock the implant to the attachment tool or, if so desired, releasing the implant via backwards motion of the corkscrew.

One method employs a removal needle with a hooking slot with its front being solid or sealed with an optional plug on the distal end of the needle allowing suction to be concentrated at the hooking slot, as in FIG. 3A and FIG. 3B, and an attachment to a suction device attached to a connector 71, as shown for example in FIG. 6A, to an external system for suction. The hooking slot may thus be used as a focal point for using suction to assist with attaching to the implant. Suction improves the attaching of the implant by the attachment tool whether it is hooking slot, corkscrew, prongs or the like.

Another embodiment of the removal method uses a removal needle with a hooking slot and attachment to a torque providing device (e.g., a drill or other rotational driver) and a force limiting fuse to prevent excessive applied torque. Unzipping of a helical wire rope structure may be achieved by untwisting it, and the removal needle with a hooking slot may be held by a torque generating device such as a drill or driver. In one embodiment there is a torque measuring and/or force limiting fuse included in-line with the torque generator and the removal needle with a hooking slot to ensure that the applied torque on the helical wire rope structure is not high enough to rip or cut it. Excessive pulling force is that which exceeds the tensile strength of the implant, particularly a helical wire rope structure. In one embodiment, the helical wire rope structure is configured to withstand seven Newtons of pulling force before being at risk of breaking, and so the force limiting fuse (in this case) is set to interrupt pulling at five to six N. In other embodiments, the helical wire rope structure may withstand 30 N and the pulling force needs to be limited to, e.g., 25 to 29 N.

A related method comprises use of a corkscrew 54 attached to a rod 54A within a removal needle 33 and an electrical driver (not depicted) to aid with twisting the corkscrew from outside the body. The corkscrew 54 may also be a corkscrew with a lead wire or a rod 54A to achieve the mechanical transfer of pulling forces from the outside of the body once the corkscrew is mechanically connected with the implant. All components may have (e.g., radiopaque) elements that improve visibility under fluoroscopy or ultrasound visualization while inside a tissue or body. In guiding a removal needle to the location of the implant, the corkscrew twists with the helical wire rope structure and then allows for the application of a pulling force to the corkscrew which transfers the removal forces into the helical wire rope structure for its release from the tissue and subsequent removal of the combination.

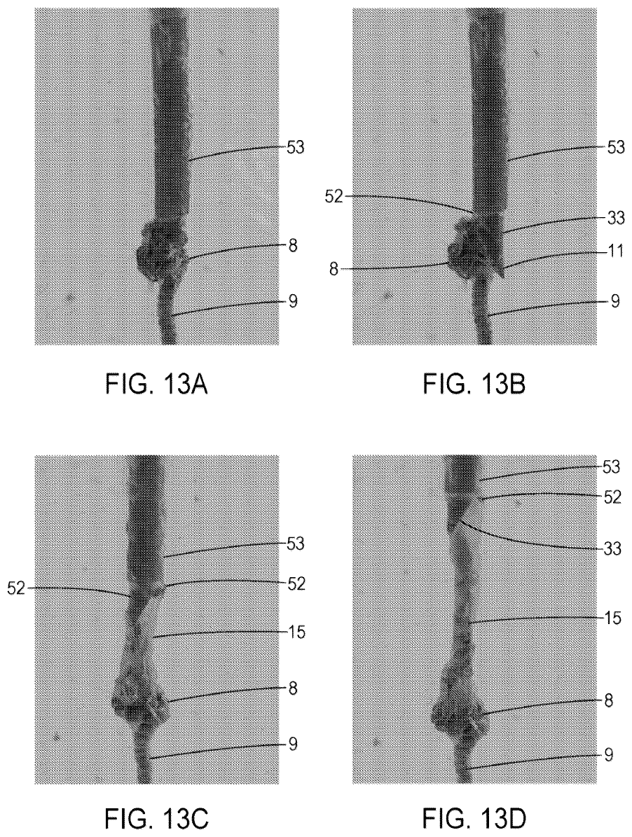

Other methods of removing an implant include:
1. Using ultrasound visualization to locate the implant and pushing a removal needle with a hooking slot to the location of the implant, moving past the implant and pulling back slightly to hook onto the implant. The devices described in this document may provide sufficient contrast under ultrasound visualization.
2. Using fluoroscopy visualization to locate the implant and pushing a removal needle with a hooking slot to the location of the implant, moving past the implant and pulling back slightly to hook onto the implant. The devices described in this document may provide sufficient contrast under fluoroscopic visualization.
3. Aiming a removal needle with a hooking slot at a bunching anchor 8 (as shown in FIGS. 13A-13D) which is easier to locate from the outside of the body, optionally without the aid of fluoroscopic or ultrasound visualization. Piercing all or a portion of a bunching anchor 8 of a helical wire rope structure on the one hand allows for an easier target to locate, pierce and mechanically hook to a helical wire rope structure, while the unzipping around the corner and in both directions allows for the removal to begin from the anchor point just as well as at a location along the substantially linear portions of the helical wire rope structure inside the body.
4. Utilizing a corkscrew as described in this disclosure to connect to and remove a helical wire rope structure.

Additional methods of removal include hooking and pulling (applying pulling force), hooking and twisting (applying torque), hooking and twisting while pulling (combination of applying pulling force and torque), and hooking and RF ablating of tissue up to a tissue depth of less than 1 mm measured radially from the helical wire rope structure and removal needle with a hooking slot before pulling (and/or twisting) the helical wire rope structure out of the tissue. Moreover, any of the methods disclosed in PCT/US21/33007 may be combined with the system, devices and method disclosed in the present application. The helical wire rope structure may be hooked from virtually any direction as long as the hook is able to mechanically interface sufficiently with the helical wire rope structure to initiate a twisting and/or pulling procedure. The pulling may be in the direction of the longitudinally greatest dimension of the helical wire rope structure (e.g., along its path through the tissue) or it may be at a direction perpendicular to the direction of the longitudinally greatest dimension of the helical wire rope structure (such as at a 90 degree angle against its path through the tissue), or at any angle in-between as the helical wire rope structure is able to unzip around a helical wire rope structure implanted and left in the tissue as having soft comer or L-shape without damaging the tissue.

The device embodying the system may be configured for one handed use so that the user may move or orient the patient with one hand while the other hand holds the device. The attachment rod 78 holding the introducer tool 64, 64A at a relative location along the length of the removal needle 33 and locked or unlocked with element 77 aids in the one handed operation of the device. The device may be easily steered to the implant with a minimal or no need for additional visualization via fluoroscopy and/or ultrasound. Instead, the introducer tool and/or removal needle may be placed blindly into the patient, aiming for the general direction of the implant, based upon the clinician's knowledge of the location.

There may be a small motor inside the handle 57 to facilitate the attachment tool connecting with the implant, or provide additional pulling and/or twisting to aid with the removal process.

FIG. 4A depicts one embodiment of a removal needle where the attachment tool is a rounded point 82 for attaching to the implant (as in FIG. 4C), particularly to a helical wire rope structure, and one embodiment of the removal needle handle 57, which can have a depth stop. The removal needle 33, removed from the removal needle handle, is shown in FIG. 4B.

FIG. 5A shows detail of a removal needle 33 and the attachment tool with rounded points 82 and cutouts 59 for capturing an implant 1. FIGS. 5B and 5C show the removal needle inside a sheath 53 and the attachment tool engaging the implant.

FIG. 6A shows an embodiment of a removal needle 33 with a side port 61 and a connection 65 to an external system, such as for suction. FIG. 6B is an enlarged view of the side port 61 and the corkscrew 54 which is the attachment tool at the end of a rod 54A, near the implant 1. FIG. 6C shows the implant captured by the corkscrew after which it will be pulled into the sheath for removal, in one embodiment at least partially assisted by the suction from the side port. FIG. 6D shows a corkscrew 54 as the attachment tool at the end of removal needle, with a connection 74, such as a weld, and a portion of the helical wire structure 15 being pulled into the corkscrew and eventually into the removal needle.

FIG. 7A is a side view showing an introducer tool with three hooked prongs 62 at the end of a rod 54A and exposed by pullback of the needle 53. At transverse section A-A, FIG. 7B shows the removal needle to which the prongs are attached, and FIG. 7C shows the long arms of the three prongs at transverse section B-B.

FIG. 8A is a side view showing a removal needle 33 with two prongs 63 with rounded edges and ends to prevent cutting an implant. At longitudinal section C-C, FIG. 8B shows the rounded prongs 63 attached to a rod 54A inside the removal needle 33, and FIG. 8C shows the prongs after they have been exposed by withdrawal of the needle, or the pulling back of the rod.

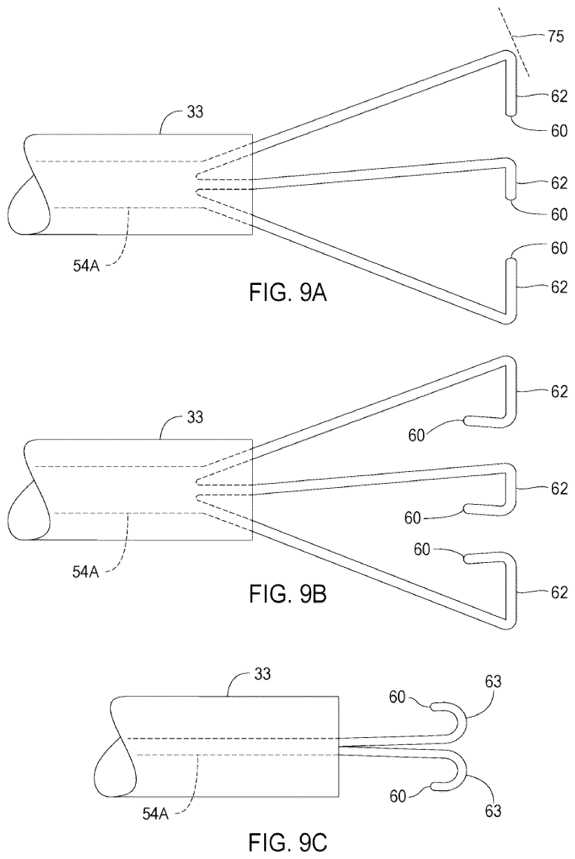

FIG. 9A is a side view showing the rounded ends 60 of a three pronged 62 attachment tool (prongs expanding when no longer compressed by a surrounding sheath) and attached to a rod 54A inside the removal needle. FIGS. 9A and 9B. show the rounded ends of the prongs, and dotted line 75 in FIG. 9A is an axis at 90 degrees to the long arms of the prongs. The angle shown, approximately 45 degrees, may be decreased (or increased) considerably to improve the ability to hook the implant. FIG. 9B illustrates that the prongs can have a third arm so that the prongs are inwardly facing hooks which expand when outside the sheath but, when pulled back, close down on the implant. FIG. 9C is a perspective schematic of the rounded ends of the attachment tool with outwardly facing hooks 63.

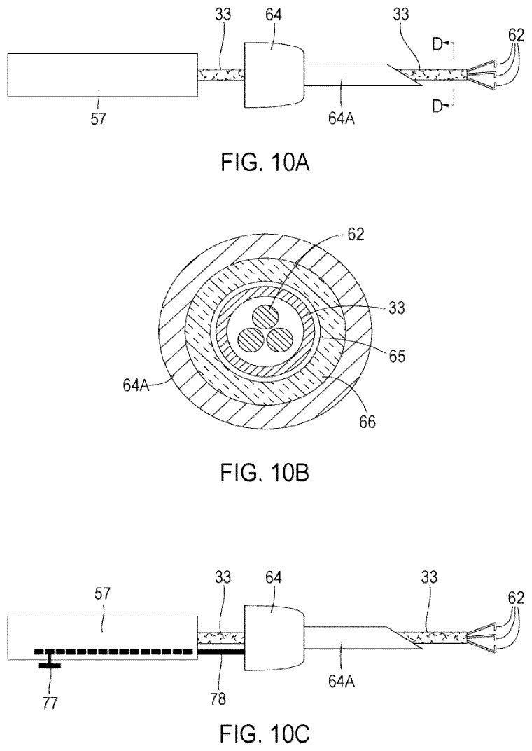

FIG. 10A shows an embodiment of the removal needle with three rounded prongs inserted through an introducer tool 64, 64A and a transverse section line D-D. When the attachment tool is inside the introducer tool 64A, FIG. 10B is the transverse section view at line D-D showing the prongs 62, needle 33, insulation 66 and introducer needle 64A. FIG. 10C shows an introducer tool engagement rod 78 linking the introducer tool to the handle of the removal needle with a lock release button 77. The introducer tool may be permanently attached to a rod which can mechanically engage with the handle of the removal needle.

Alternatively, the introducer tool may be temporarily attached to a rod (needle), the rod itself ending in a Luer Lock to attach to needle on one (here: the right) side and as a rod on the other (left) side which can mechanically engage with the handle 57 of the removal needle.

The image shows the needle screwed onto the Luer Lock, thereby mechanically attached to the rod. Once the rod is introduced into the handle 57, it locks in place and prevents the introducer tool from falling off the shaft of the removal needle. Upon pushing the release button, the rod may either move in or out of the handle. With the removal needle in the locked position protruding forward and covering the shaft of the removal needle, the operator punctures a hole into the skin of a body and then pushes the release button to enable the shaft to move freely through the introducer tool (as the rod slides into the handle) and perform a blunt access through the tissue between the tip of the introducer tool and the implant.

The force to rip native connective tissue captured within the attachment tool is greater than the force to remove the helical wire rope structure. When manually applying a pulling force, a user will first feel the resistance of the tissue and not the implanted device. The difference between tissue ripping force and implant removal force causes an abrupt change in force applied by the user, where the applied native tissue ripping force has an impact on the force applied on the helical wire rope structure during subsequent removal.

FIG. 11 shows a combination of a force limiting fuse and a force smoothing device of the system, with a spring socket 69 and ball 70 attached by a connector 71 to the extension limiting canister 72 with the smoothing spring 73 connected at point 74 to the attachment tool (not pictured). At a predetermined release force, the ball will release from the socket to provide a set force limit that is easily reset by plugging the ball back into the socket. The difference in length of the spring and length of the canister provide the extension limit for the smoothing spring, with the spring dictating the degree of force smoothing. The force limiting fuse and the force smoothing device are here depicted together, but each can be used separately by itself in a different embodiment. In one embodiment, the device depicted in FIG. 11 may be placed inside the removal needle between the transverse section cut of the removal needle 53 at line A-A and the transverse section cut at line B-B in figure FIG. 7B. In another embodiment, the device depicted in FIG. 11 may be placed between the transverse section cut of the removal needle at line A-A in figure FIG. 7B and the hand piece.

Another embodiment of the method herein comprises the attachment tool with an attached force limiting and smoothing system (in FIG. 11), ensuring that the rate of change in force is gradual and limited. The force limiting and smoothing devices comprise respectively a mechanical fuse 69, 70 and an expansion limited spring 73, which provide force limiting and smoothing respectively. The smoothing spring 73 delays the transition of force from the removal needle as held by the user and the hooked or corkscrew needle when a linear force is applied parallel to the central axis of the removal needle. Limiting the extension of the spring creates a maximum smoothing range, which when fully extended to a predetermined length transitions to direct force application without smoothing. In one example, the tissue ripping force is 10 N, the removal force is 5 N, and helical wire rope structure failure force is 15 N, with a total removal length of 200 mm. The mechanical force limiting fuse is set at a value above the tissue ripping force and below the helical wire rope structure failure force. The smoothing spring should have a spring constant high enough to provide sufficient resistance to allow force transfer from the user to the needle, but low enough to provide a smoothing effect. The helical wire structure in this example is removed at approximately 10 mm/s at a maximum force of 12 N as set by the fuse. The force transition from tissue ripping to removal occurs in under 1 second, so a maximum of 10 mm of needle movement is to be expected, with a transition from 12 N to 5 N. With this example case, an appropriate smoothing spring would have a spring constant between 0.5 to 1.2 N/mm, with the higher values providing less smoothing. The maximum extension of the spring would be limited to 10 mm. The function of the smoothing spring is to smooth transient spikes in force from the operator's hand to the helical wire rope structure. As a result, the maximum force translated to the helical wire rope structure is lower.

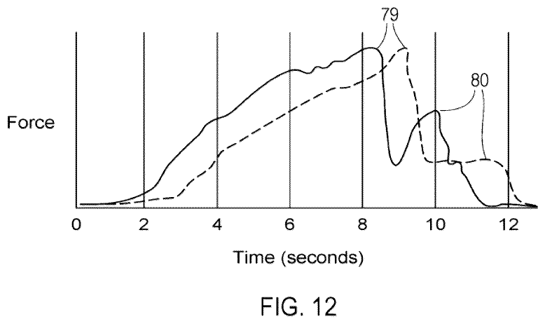

FIG. 12 is a chart showing measured removal force (in Newtons) over time without (solid) and with a smoothing device (broken line), with maximal native tissue ripping force 79 and maximum implant removal force 80.

FIGS. 13A-13D are a sequence of images showing removal of a helical wire rope structure where, similar to the embodiments in FIGS. 1A-1G, the attachment tool is a hooking slot 52.

FIG. 14 is a schematic of the wire hook tool 67 with a wire and a connector 75 to a hook 68.

The system disclosed herein may enable the minimally invasive placement of a self-anchoring helical wire rope structure into a body, using a small diameter needle of 10 to 20 Gauge, without a need for sutures to secure it or to close the puncture wound. This implant may be left in place acutely or chronically for days, months or years, after which it may be removed using a small diameter needle of 10 to 20 Gauge to puncture an opening into the tissue through which this implant may be removed by unzipping and without portions of the helical wire rope structure being left behind. There is no need for an open cut down to gain access to the anchor portion of this implant and no need to suture the puncture wound created with the introducer needle or the removal needle due to their small diameter size.

We claim:

1. A method of removing an implant from bodily tissue, comprising the steps of: advancing an introducer tool to the location of the implant;
inserting a removal needle having an attachment tool through the introducer tool;
mechanically engaging the attachment tool with the implant; and pulling out the implant with the removal needle.

2. The method of claim 1 wherein said step of pulling out the implant with the
removal needle is performed by pulling the removal needle through the introducer tool while the introducer tool stays in place as a channel.

3. The method of claim 1 wherein said step of pulling out the implant with the removal needle is performed by pulling a portion of the implant into the removal needle and thereafter pulling the introducer tool out of the body.

4. The method of claim 1 wherein said step of mechanically engaging the attachment tool with the implant comprises at least one step selected from the group of:
hooking the attachment tool onto the implant and pulling the attachment tool;
hooking the attachment tool onto the implant and twisting the attachment tool;
hooking the attachment tool onto the implant and twisting while pulling the attachment tool; and
hooking the attachment tool onto the implant and using RF ablation of the tissue before pulling.
